(19) United States
(12) Patent Application Publication
(10) Pub. No.: US 2022/0175878 A1
(43) Pub. Date: Jun. 9, 2022

(54) METHOD FOR PREPARING EYE DROPS OF CYCLOSPORIN A

(71) Applicant: CHU CLERMONT-FERRAND, Clermont-Ferrand (FR)

(72) Inventors: Philip CHENNELL, Aubiere (FR); Frederic CHIAMBARETTA, Clermont-Ferrand (FR); Lucie DELABORDE, Clermont-Ferrand (FR); Mireille JOUANNET, Clermont-Ferrand (FR); Valerie SAUTOU, Riom (FR)

(21) Appl. No.: 17/682,094

(22) Filed: Feb. 28, 2022

Related U.S. Application Data
(63) Continuation of application No. 15/762,688, filed on Mar. 23, 2018.

(30) Foreign Application Priority Data
Oct. 2, 2015 (FR) 1559367

(51) Int. Cl. A61K 38/13; A61K 9/00; A61K 9/107; A61K 47/44; A61K 31/728; A61K 31/79

(52) U.S. Cl. CPC A61K 38/13; A61K 9/0048; A61K 9/1075; A61K 31/79; A61K 31/728; A61K 9/107; A61K 47/44

(57) ABSTRACT

Eye drops containing cyclosporin A are prepared in the form of an emulsion, a microemulsion, or a lipophilic/hydrophilic micellar solution whose lipophilic, and hence oily, phase contains cyclosporin A. Preparation of the eye drops begins by eliminating at least 99 wt % of ethanol contained in an oily solution of cyclosporin A, under a flow of nitrogen under a pressure of 0.5 bar, at a temperature of from 2° C. to 45° C. and for a period of from 0.25 h to 24 h. The concentrate of cyclosporine is then diluted in an aqueous solution. Next, an emulsion, a microemulsion, or a micellar solution being 75% aqueous phase is created by stirring the concentrated cyclosporine A with the aqueous solution to mix them together. The resulting mixture is sterilely packaged as eye drops in packaging that is suitable for ophthalmic use.

### BACKGROUND OF THE INVENTION

The present invention relates to eye drops of cyclosporin A.

Cyclosporins are cyclic oligopeptides having immunosuppressive and anti-inflammatory activities from the family of the anti-calcineurins. Among the cyclosporins, cyclosporin A is a molecule that is used in the formulation of some medicaments for the treatment of various inflammatory pathologies, either by general or local route. The use of eye drops of cyclosporin A 0.5 to 20 mg/ml for the treatment of chronic inflammatory pathologies of the eye such as vernal keratoconjunctivitis, dry eye syndrome that is resistant to the first line of treatment, and for the prevention or treatment of corneal transplant rejection is known in ophthalmology.

Since the cyclosporins are lipophilic, they are essentially employed in oil-based formulations. For local administration by ocular route, the oils that are commonly used as a vehicle for the active substance include but are not limited to olive oils, peanut oil, castor oil, polyoxyethylene castor oil, or mineral oils. It turns out that oil-based cyclosporin formulations exhibit low ocular tolerance, which quite frequently induces adverse effects such as eye irritation, burning sensations upon instillation, tearing, or even changes in visual quality.

F. Chast et Al, J Fr. Ophtalmol. (2004-01-01), pages 567-576, XP055283877 describes the preparation of eye drops of cyclosporin 2% with castor oil and in the presence of ethanol. The eye drops obtained are a purely oily solution. This document does not provide for the addition of a lubricating and/or wound-healing agent. It is known from WO-A-9531211 to reduce the amount of oil and form an emulsion with water. WO-A-2006/050838 discloses an oil/water emulsion for ophthalmic use comprising colloid particles coated by an interfacial film. Such a solution can be administered in form of a patch, implant, or insert but is not suitable for use in the form of eye drops. WO-A-2005/032577 describes an ophthalmic formulation for eye drops that is intended particularly for the treatment of dry eye comprising water, a hydrophobic compound, a cyclosporin A having a concentration of less than 1 mg/ml, or less than 0.1% by total weight. The hydrophobic compound is selected from among the vegetable, animal, mineral, or synthetic oils and from among mixtures of these oils. Other components such as stabilizers or surfactants are also used. In one embodiment, the formulation does not contain any preservatives. It also comprises a polyanionic compound of a surface-active nature or one that is employed as a lubricant and wound-healing agent such as hyaluronic acid, among others. If needed, preservatives are incorporated into the formulation.

It was found that it may be necessary to have a higher concentration of cyclosporin, specifically greater than or at least equal to 1 mg/ml, or 0.1% by total weight, for example between 5 mg/ml and 25 mg/ml, inclusive.

Such concentrations are necessary for the treatment of vernal keratoconjunctivitis, for example, in order to prevent and/or treat corneal transplant rejections.

And yet it turns out that such preparations are not currently available on the market as a medicament with marketing authorization (AMM) or expanded access covering all needs, particularly for the pediatric population. As a result, such preparations must be prepared in a hospital setting by Pharmacies for Internal Use (PUI), namely by pharmacy departments that are dedicated to a single hospital entity with which they are associated. Consequently, the formulations and/or conditions of preparation are not standardized among the different establishments both in France and throughout Europe. This results in substantial differences in the ocular tolerance, the conditions, and/or the shelf lives of such preparations. What is more, such preparations often comprise, in more or less significant concentrations, ethanol, preservatives, or they are entirely oily, which poses problems with tolerance of instillation in the eye, for example, and on the corneal surface over time.

It is more particularly the aim of the invention to propose a different method for preparing eye drops that enables stronger concentrations of cyclosporin A to be obtained, typically greater than 1 mg/ml, without the need for preservatives and with a minimal concentration of ethanol while offering optimized ocular tolerance.

### BRIEF SUMMARY OF THE INVENTION

To this end, it is the object of the invention to provide a method for preparing eye drops of cyclosporin A in the form of an emulsion, a microemulsion, or a lipophilic/hydrophilic micellar solution whose lipophilic—and hence oily—phase contains cyclosporin A, characterized in that it comprises at least the following steps:
a) eliminating at least 99 wt % of the ethanol contained in the oily solution of cyclosporin A, under a flow of nitrogen under a pressure of 0.5 bar, at a temperature of from 2° C. to 45° C. and for a period of from 0.25 h to 24 h,
b) diluting the concentrate obtained in step a) in an aqueous solution,
c) forming an emulsion, a microemulsion, or a micellar solution in which the aqueous phase makes up at least 75% of the final emulsion, microemulsion, or micellar solution, by stirring to mix the concentrated oily solution obtained in step a) with the diluted aqueous solution obtained in step b),
d) sterile packaging of the obtained eye drops in packaging that is suitable for ophthalmic use.

According to advantageous but optional aspects of the invention, such a method can comprise one or more of the following characteristics:
The eye drops are prepared without the addition of antimicrobial preservatives.
During step b), a solution of lubricating and/or wound-healing agent is incorporated into the aqueous solution.
The lubricating and/or wound-healing agent is selected from among hyaluronic acid and salts thereof or povidone.
The solution of lubricating and/or wound-healing agent is reconstituted from powder or is ready to use.
During step d), the eye drops are subjected to final sterilization prior to packaging.
The oily solution of cyclosporin A is a solution of cyclosporin A in hydrodispersive and emulsifying oil.
The oily solution of cyclosporin A is a solution of cyclosporin A in polyoxyethylene castor oil.
The concentration of cyclosporine A in the eye drops obtained at the end of step d) is between 1 mg/ml and 40 mg/ml, inclusive.
The concentration of cyclosporine A in the eye drops obtained at the end of step d) is between 5 mg/ml and 25 mg/ml, inclusive.
The eye drops obtained at the end of step d) are suitable for human or veterinarian use.

### DETAILED DESCRIPTION OF THE INVENTION

The invention will be better understood and other advantages thereof will become clearer from the following description of several embodiments of the invention, which are provided by way of non-limiting example.

It should be noted here that such a method is advantageously but not exclusively intended for implementation in a hospital setting.

It is known that, in many chronic inflammatory eye diseases, the use of cyclosporin in local application as an alternative to the corticoids is considered attractive. Such preparations are prepared by the Pharmacies for Internal Use, or PUIs, of healthcare establishments in observance of the regulations in force, i.e., Good Manufacturing Practices, among others, to the extent that no proprietary medicinal product that meets the needs is available or adapted to the use in question.

It will readily be understood that such a method can also be implemented in the framework of commercial production in observance of the regulations in force.

The method that is the object of the invention is particularly but not exclusively suitable for the preparation of formulations having a concentration of cyclosporin A of between 1 mg/ml and 40 mg/ml, and preferably between 5 mg/ml and 25 mg/ml.

At present, the preparations that are prepared at hospital centers are mostly prepared solely with oil. Consequently, the cyclosporins have poor solubility in water. As an example, the solubility of cyclosporin A is on the order of 20 to 30 μg/ml. In other words, when greater concentrations of cyclosporin A are required—typically on the order of 300 to 1000 more, and hence concentrations of about 10 mg/ml to 20 mg/ml—an aqueous solution of cyclosporin A cannot be easily achieved.

While the use of a formulation that is solely oil-based or based on a mixture of oils does mitigate this problem, it produces problems with tolerance. It is known from the prior art (Yavuz B et al; An Overview on Dry Eye Treatment: Approaches for Cyclosporin A Delivery; The Scientific World Journal, 2012; 2012:1-11. Lallemand F, et al; Cyclosporine A delivery to the eye: A pharmaceutical challenge; European Journal of Pharmaceutics and Biopharmaceutics, nov 2003; 56(3):307-18. H. Nourry et al. 2006, Étude de la cytotoxicité de différents collyres à base de ciclosporine A buvable (Sandimmun®) [Study of the cytotoxicity of different eye drops based on drinkable cyclosporin A (Sandimmun®)], Journal Francais d'Ophtalmologie, March 2006; 29(3):251-7) that formulations that are based only on oil induce burns, tingling, and pain that can last for several hours after the application of the eye drops.

By virtue of a preparation that combines a lipophilic compound and a hydrophilic compound, typically a lipophilic/hydrophilic emulsion or a lipophilic/hydrophilic microemulsion or a micellar solution whose dispersed phase is lipophilic and contains cyclosporin A, with the continuous phase of this micellar solution being aqueous, it is possible to obtain eye drops the tolerance of which is improved compared to an exclusively lipophilic compound, all while retaining superior solubility, and thus superior feasibility, compared to preparations that are solely aqueous.

It is common to incorporate a preservative in order to enable the shelf life of these preparations to be optimized, for example after the packaging is opened. The preservative, or a mixture of preservatives, is selected from among the quaternary ammonium compounds, mercury derivatives, alcohols, and chlorhexidine salts, to name a few non-limitative examples. It is known and accepted that preservatives are potentially toxic to the corneal endothelium, in part due to their direct cytotoxicity, and in part due to their detergent effect, which alters the lacrimal film and causes ocular dryness. These problems are particularly present when quaternary ammonium compounds are used as preservatives. Some noteworthy examples of preservatives include but are not limited to benzalkonium chloride, cetrimide, polysorbate, EDTA or ethylenediaminetetraacetic acid, chlorobutanol, and potassium perborate.

The use of a preparation without antimicrobial preservatives enables eye drops to be obtained that are better tolerated compared to eye drops containing them.

Advantageously but optionally, the aqueous phase of such a preparation makes it possible to incorporate additives to the preparation in the sense of additional active substances. Preferably, the aqueous phase enables the incorporation of a lubricating and/or wound-healing compound. These include but are not limited to hyaluronic acid/sodium hyaluronate, povidone, carbomers, polyvinyl alcohol, polyvidone, dextrans, polysaccharides, the family of carboxy or methyl or propyl cellulose, glycerin, or glycerol. It is known, for example from EP-A-1142566, to improve the bioavailability and tolerance of an oil/water emulsion of cyclosporin by incorporating hyaluronic acid or a salt thereof. This enables the viscosity of the eye drops to be increased, thus promoting the good distribution of the eye drops on the surface of the eye and improving their efficacy. This addition is also used for its lubricating and/or wound-healing properties, which improves the tolerance on instillation and the stability of the lacrimal film.

The present invention relates to an ophthalmic preparation of the lipophilic/hydrophilic emulsion or lipophilic/hydrophilic microemulsion or micellar solution type in which the dispersed phase is lipophilic and contains cyclosporin A, with the continuous phase of this micellar solution being aqueous, in a concentration of at least 1 mg/ml, with no preservatives and with a lubricating and/or wound-healing agent.

In particular, as non-limitative examples, the applicant prepared two formulations of the water emulsion type without preservatives with two different lubricating and/or wound-healing agents, each in three different concentrations of cyclosporin A. The following examples are given for the concentrations of 1, 10, and 20 mg/ml. The invention conceivably enables any concentration of cyclosporin A to be achieved from 1 to 40 mg/ml, inclusive.

### Example 1

Preparation with Hyaluronic Acid as a Lubricating and Wound-Healing Agent.

The hyaluronic acid is either in the form of an acid or in the form of a salt such as a sodium hyaluronate, a potassium hyaluronate, or a calcium hyaluronate.

A mixture of hyaluronic acid and sodium hyaluronate in aqueous solution (water for injection or WFI) and, optionally, sodium chloride, mono- and disodium phosphate, as well as hydrochloric acid is advantageously used. The mixture is ready to use or is prepared freshly with dissolution of the various compounds under stirring.

The oil is selected from among a vegetable, animal, mineral, or synthetic oil that preferably has hydrodispersive and emulsifying properties, ensuring the solubility of the lipophilic compounds while enabling the emulsion to be created. In a variant, it is a microemulsion or a micellar solution. Some noteworthy examples of compounds of an oily nature are glycerol, polysorbate 80, and synthetic glycerides.

Among the synthetic glycerides, a polyoxyethylene castor oil is preferably used with a molar ratio of between 25 and 50, inclusive, and preferably between 35 and 45.

Table 1 below shows the detailed formulations of one method of preparation for cyclosporin concentrations of 1, 10, and 20 mg/ml.

Here, the lubricating and wound-healing agent is based on hyaluronic acid.

TABLE 1

| | Formulation 1 | | | | | |
|---|---|---|---|---|---|---|
| Cyclosporin | 1 | mg/ml | 10 | mg/ml | 20 | mg/ml |
| Polyoxyethylene castor oil | 13 | mg/ml | 130 | mg/ml | 260 | mg/ml |
| Ethanol | 0-5.56 | mg/ml | 0-5.56 | mg/ml | 0-11.12 | mg/ml |
| Dissolved nitrogen | Possible traces | | Possible traces | | Possible traces | |
| Aqueous solution (WFI) | Required quantity | | Required quantity | | Required quantity | |
| of which: Hyaluronic acid/ | 1.47 | mg/ml | 1.305 | mg/ml | 1.125 | mg/ml |
| sodium hyaluronate | | | | | | |

of which: Sodium chloride, mono- and disodium phosphate, hydrochloric acid

| | | | |
|---|---|---|---|
| % of oily phase | 2% | 12.50% | 25% |
| % of aqueous phase | 98% | 87.50% | 75% |

A variation of plus or minus 10% of the indicated target concentration values is acceptable.

The concentration of cyclosporin A is measured either by visible UV spectrophotometry or by inversed-phase high-performance liquid chromatography.

Note that, regardless of the concentrations of cyclosporin, the residual alcohol content—ethanol in this case—is low, namely less than 12 mg/ml. In addition, the residual ethanol content is controlled. The oily solution of cyclosporin used as a raw material is either ready to use or prepared specifically and contains polyoxyethylene castor oil and ethanol.

In other words, this is a solution of cyclosporin in hydrodispersive and emulsifying oil, such as polyoxyethylene castor oil, for example, without any other oil or agents, and thus without polysorbate or surfactants. Only the ethanol is present. The concentration of ethanol in the oily solution of cyclosporin that is used as a raw material can reach 278 mg/ml.

The presence of ethanol makes it possible to improve the solubility of the cyclosporin in the oily phase, but it is unfavorable for ocular tolerance due to pro-inflammatory and irritant effects on the cornea. It must therefore be removed after solubilization of the cyclosporin in the polyoxyethylene castor oil in order to obtain eye drops having good tolerance on instillation.

### Example 2

The following table shows the detailed formulations of another method of preparation for cyclosporin concentrations of 1, 10, and 20 mg/ml in which the lubricating and wound-healing agent is povidone-based.

Povidone is an ocular lubricant. It is a synthetic polymer containing linear chains of 1-vinyl-2-pyrrolidone. It temporarily compensates for the lack of tears. Advantageously, it is used in the form of a ready-to-use solution without preservatives or in the form of powder that is diluted freshly in sterile, pyrogen-free water (WFI) or balanced saline solution (BSS).

Like in the first example, the oil that is preferably used is polyoxyethylene castor oil.

Table 2 below shows the detailed formulations of such a method of preparation for cyclosporin concentrations of 1, 10, and 20 mg/ml.

TABLE 2

| | Formulation 2 | | | | | |
|---|---|---|---|---|---|---|
| Cyclosporin | 1 | mg/ml | 10 | mg/ml | 20 | mg/ml |
| Polyoxyethylene castor oil | 13 | mg/ml | 130 | mg/ml | 260 | mg/ml |
| Ethanol | 0-5.56 | mg/ml | 0-5.56 | mg/ml | 0-11.12 | mg/ml |
| Nitrogen | Possible traces | | Possible traces | | Possible traces | |
| Aqueous solution (WFI) | Required quantity | | Required quantity | | Required quantity | |
| of which: povidone | 14.7 | mg/ml | 13.125 | mg/ml | 11.25 | mg/ml |

of which: sodium chloride, sodium acetate, sodium citrate, calcium chloride, magnesium
chloride hexahydrate, potassium chloride, sodium hydroxide, hydrochloric acid.

| | | | |
|---|---|---|---|
| % of oily phase | 2% | 12.50% | 25% |
| % of aqueous phase | 98% | 87.50% | 75% |

A variation of plus or minus 10% of the indicated target concentration values is acceptable.

The concentration of cyclosporin A is measured either by visible UV spectrophotometry or by inversed-phase high-performance liquid chromatography.

Note that, here as well, similarly to example 1, the residual alcohol content—ethanol in this case—is low, namely less than 12 mg/ml, and controlled regardless of the concentrations of cyclosporin.

In the two examples, note that the proportion of oil in the emulsion varies from 2% to 25%. These proportions are necessary for the solubilization and the stability of the emulsion or microemulsion, since they enable the precipitation of the cyclosporin A to be prevented.

The preparation of these eye drop formulations will now be given for a case of eye drops containing hyaluronic acid, with it being understood that the preparation is identical when the eye drops comprise povidone.

The first step consists in eliminating at least 99 wt % of the alcohol present in the oily solution of cyclosporin—ethanol in the present case. This elimination is achieved under the following conditions, for example:

At a controlled temperature that is no greater than 45° C., and advantageously about 25° C., a nitrogen flow under a pressure of 0.5 bar is aimed at and concentrated by means of a nozzle onto the surface of the oily solution to be treated. The treatment is applied for the necessary duration, with it being understood that this period is no less than 14 hours.

Nitrogen is a neutral gas that does not provoke chemical changes in the other components of the formulation.

It is also possible to eliminate the ethanol by other inherently known techniques, for example through passive evaporation to the ambient air or under a vacuum.

Nevertheless, the use of a nitrogen flow is a preferred evaporation technique, since it is easy to perform and control while also being faster than passive evaporation. By way of example, the final concentration of ethanol is less than 11.12 mg/ml when eye drops are prepared having a concentration of cyclosporin of 20 mg/ml.

The next step consists in diluting, in aqueous solution, the concentrate of cyclosporin in the oily solution obtained in the previous step. Advantageously, an aqueous solution of ocular lubricant/wound-healing agent is introduced during this step. This is a mixture of hyaluronic acid and sodium hyaluronate. The aqueous solution containing the lubricant/wound-healing agent is either ready to use or prepared just before use. In this case, the solution is advantageously prepared from a powder of the desired lubricant/wound-healing agent and WFI. In all cases, the conformity of the solution is checked by the pharmacy for internal use. Mixing is advantageously performed at room temperature, advantageously at 25° C., and under germ-free or at least so-called clean conditions.

The following step consists in incorporating the aqueous phase obtained in this way and preparing the emulsion, microemulsion, or micellar solution. To do this, the aqueous solution obtained in the previous step is mixed with the concentrate of cyclosporin A in oily solution that is available at the end of the first concentration step. The mixture of the two lipophilic and hydrophilic phases is produced through the application of kinetic energy by stirring the solution, for example with the aid of a magnetic stirrer or another inherently known technique. In this way, all of the oily fraction containing the cyclosporin is dispersed in the aqueous phase under stirring in order to obtain an emulsion, microemulsion, or micellar solution, with it being understood that the aqueous phase represents at least 75% of the final emulsion, microemulsion, or micellar solution here.

The mixing under stirring is performed at a temperature of between 2° C. and 45° C., inclusive, for a period of from 0.05 h to 48 h. It should be noted that the micellar solution obtained is more precisely an aqueous micellar solution in which the size of the majority of the colloidal micelles present is less than 25 nm.

Once the eye drops have been prepared, steps should be taken in the last step to package the eye drops in such a way as to ensure the sterility of the preparation. The finished preparation undergoes final sterilization at the time of packaging in a controlled atmosphere; this can be achieved through sterilizing filtration, for instance.

Due to the absence of preservatives, packaging that makes use of the “multidose without preservatives” technology that maintains the sterility of the eye drops after the sterile, single-dose container or packaging is opened is advantageously required. The eye drops are thus advantageously presented in the form of an identified packaging for the specific patient that contains a defined concentration of cyclosporin depending on the pathology to be treated.

The eye drops can conceivably be presented in the form of a kit comprising several single doses for a treatment over a limited number of days and/or a given number of patients. In a variant, the eye drops are presented in the form of a multidose vial that guarantees that the sterility is maintained after opening. Such a vial can be used for a defined and validated number of days, such as 30 days, for example.

Tests were conducted in order to check the stability of the preparation throughout the defined keeping time before and after opening, which enabled the conditions for storage before and after opening to be defined.

Table 3 shows the data obtained during the storage tests at a temperature of 4° C. and at a temperature of 25° C. The applicant did not observe a significant difference in stability between the two storage temperatures over a storage period of 90 days. Additional studies are in progress and are aimed at extending the storage life of the eye drops that are the object of the invention.

All of these tests are being conducted in compliance with the recommendations in force.

TABLE 3

| Concentration of | | | |
|---|---|---|---|
| cyclosporin A | 1 mg/ml | 10 mg/ml | 20 mg/ml |
| Maximum storage time | 90 days | 90 days | 90 days |
| Storage after opening | 30 days | 30 days | 30 days |

On the other hand, the quality of the final preparation is verified for each lot of eye drops produced through the representative sampling of the lot. In order to ensure that the preparation satisfies the regulatory requirements of the European Pharmacopoeia.

The statutory labeling of the preparation is also performed in order to identify the different concentrations of cyclosporin and/or the conditions of use and/or for storage.

In a variant, a visual identification sign is used for the different concentrations of cyclosporin.

It will readily be understood that such a preparation is suitable not only for human but also veterinary use.

We claim:

1. A method of preparing an eye drop formulation without preservatives, comprising:
a) eliminating at least 99 wt % of ethanol contained in an oily solution, said oily solution comprising cyclosporine A, the ethanol, and polyoxyethylene castor oil, under a flow of nitrogen, under a pressure of 0.5 bar, at a temperature of from 2° C. to 45° C., and for a time period of 14 h to 24 h to form a concentrate of cyclosporine A in the oily solution,
b) diluting the concentrate of cyclosporine A in the oily solution obtained in step a) in an aqueous solution,
c) mix under stirring the concentrated oily solution obtained in step a) with the aqueous solution obtained in step b), at a temperature of between 2° C. and 45° C. for a period of from 0.05 h to 48 h to obtain a micellar solution having colloidal micelles less than 25 nm in size, and
d) sterily packaging the micellar solution obtained in step c) for use as the eye drop formulation in packaging that is suitable for ophthalmic use.

2. The method of claim 1, wherein a lubricating and/or wound-healing agent is added to the aqueous solution in step b), before step c).

3. The method of claim 2, wherein the lubricating and/or wound-healing agent is reconstituted from a powder or is a liquid solution.

4. The method of claim 1, wherein, during step d), the eye drop formulation is subjected to a final sterilization prior to packaging.

5. An eye drop formulation suitable for human or veterinary use prepared by the method of claim 1, comprising:
the micellar solution having
an oily phase containing the cyclosporin A, the oily phase having a concentration of cyclosporine A between 5 mg/ml and 25 mg/ml;
the polyoxyethylene castor oil with a molar ratio between 25 and 50;
the ethanol in a concentration less than 12 mg/ml in the micellar solution; and
a lubricating and wound-healing agent based on a hyaluronic acid, without the preservatives.

6. The eye drop formulation of claim 5, wherein the polyoxyethylene castor oil has a molar ratio between 35 and 45.
